(19) United States
(12) Patent Application Publication
(10) Pub. No.: US 2013/0110149 A1
(43) Pub. Date: May 2, 2013

(54) SYNTHETIC GEMSTONES SUITABLE FOR CONTACT WITH HUMAN TISSUE AND METHODS OF USE THEREOF

(71) Applicant: Fritz Schomburg, Madison, WI (US)

(72) Inventor: Fritz Schomburg, Madison, WI (US)

(21) Appl. No.: 13/661,790

(22) Filed: Oct. 26, 2012

Related U.S. Application Data
(60) Provisional application No. 61/552,988, filed on Oct. 28, 2011.

(51) Int. Cl. A44C 7/00; A61B 17/34

(52) U.S. Cl. 606/188; 63/12

(57) ABSTRACT

The present invention provides synthetic gemstones for use in contact with human tissue, compositions, devices and systems comprising such gemstones, and methods of use thereof.

This application claims priority to provisional patent application Ser. No. 61/552,988, filed Oct. 28, 2011, which is herein incorporated by reference in its entirety.

### FIELD

The present invention provides synthetic gemstones for use in contact with human tissue, compositions, devices and systems comprising such gemstones, and methods of use thereof.

### BACKGROUND

Objects, particularly metallic objects, which are in direct and continuous contact with human skin and other tissues (e.g., jewelry (e.g., earrings, arm bands, necklaces, body piercings, tissue stretchers, etc., implants, dental work, etc.) often cause allergic or other adverse reactions on and/or in the skin or tissue and spreading out from the point of contact. The consequence of these reactions is typically in the form of itching, wet skin sores, and/or painful rash of the skin. In some instances the reactions may affect the organism generally, and may lead to disorders of various parts of the body. In addition, porous objects that are placed in contact with human skin or tissues can provide venues for bacterial growth, and may present a potential infection risk.

### SUMMARY

In some embodiments, the present invention provides compositions comprising synthetic gemstone material, wherein the composition is configured to place the synthetic gemstone material in direct and continuous contact with human or animal skin or tissue and to avoid contact between other materials of the composition and the human or animal skin or tissue. In some embodiments, the region of direct and continuous contact comprises at least 0.1 cm² of surface area. In some embodiments, the region of direct and continuous contact comprises at least 0.25 cm² of surface area. In some embodiments, the region of direct and continuous contact comprises at least 1 cm² of surface area. In some embodiments, the duration of direct and continuous contact comprises at least 1 hour. In some embodiments, the duration of direct and continuous contact comprises at least 4 hours. In some embodiments, the the duration of direct and continuous contact comprises at least 1 day (e.g., at least 2, 3, 4, 5, . . ., 10, . . ., etc. days). In some embodiments, the composition consists entirely of synthetic gemstone material. In some embodiments, the composition is jewelry, a medical implant, a dental implant, an entertainment device, or a communication device. In some embodiments, the composition is configured to contact the surface of the skin of a human or animal. In some embodiments, the composition is configured for insertion into a fistula of the skin of a human or animal. In some embodiments, the composition is configured for insertion into the tissue of a human or animal.

In some embodiments, the present invention provides methods of preventing adverse reactions to an object that is placed in direct and continuous contact with human or animal skin or tissue comprising manufacturing the object to be configured to place the synthetic gemstone material in direct and continuous contact with human or animal skin or tissue and to avoid contact between other materials of the composition and the human or animal skin or tissue. In some embodiments, the object consists entirely of synthetic gemstone material. In some embodiments, the object contacts the surface of the skin of a human or animal. In some embodiments, the object is inserted into a fistula of the skin of a human or animal. In some embodiments, the object is inserted the tissue of a human or animal. In some embodiments, the object is: a piece of jewelry, a medical implant, a dental implant, a communication device, or an entertainment device.

In some embodiments, the present invention provides methods of preventing adverse reaction to an object that is placed in direct and continuous contact with human or animal skin or tissue comprising replacing the object with a composition as described herein.

### BRIEF DESCRIPTION OF THE DRAWINGS

FIG. 1 shows an illustration of an exemplary body piercing plug with a cylinder-like shape that is narrowed in the middle and flares out at either end.

### DETAILED DESCRIPTION

In some embodiments, the present invention provides synthetic gemstones (e.g., ruby, sapphire, emerald, aquamarine, red beryl, goshenite, heliodor, morganite, etc.) and compositions, devices, objects, etc. made thereof configured for direct and/or continuous contact with human skin and/or tissue. In some embodiments, compositions, devices, objects of the present invention consist only of synthetic gemstone and do not contain other materials such as metals (e.g., to prevent allergic reaction-causing contact with non-gemstone material, to prevent providing a vector for bacterial growth, etc.). In some embodiments, compositions, devices, objects of the present invention comprise synthetic gemstone and one or more other materials, but are designed, configured, shaped, etc. to place only the synthetic gemstone material, and not other materials (e.g., potentially allergic reaction causing materials, metals, etc.), in contact (e.g., direct and continuous contact) with the skin or other tissues. In some embodiments, compositions, devices, objects of the present invention are designed, configured, shaped, etc. to minimize skin and/or tissue contact with non-gemstone materials.

Different processes have been developed over the years for the synthetic production of synthetic gemstones and their underlying materials (Bahadur: a Handbook of Precious Stones. 1943. Retrieved 2007 Aug. 19.; herein incorporated by reference in its entirety). The production of synthetic rubies, sapphires, emeralds, aquamarine, etc. is well understood in the field, see, for example U.S. Pat. No. 3,950,596; U.S. Pat. No. 3,088,194; U.S. Pat. No. 2,488,507; U.S. Pat. No. 2,690,062; U.S. Pat. No. 3,539,379; U.S. Pat. No. 4,039,726; U.S. Pat. No. 6,475,942; U.S. Pat. Pub. No. 2002/0135108; U.S. Pat. No. 6,572,670; U.S. Pat. No. 4,476,090; U.S. Pat. No. 4,543,342; U.S. Pat. No. 4,835,023; U.S. Pat. No. 4,030,317; all of which are herein incorporated by reference in their entireties.

In some embodiments, synthetic gemstones (e.g., rubies) are produced by the boule, oxyhydrogen-flame, or Verneuil process; or variations thereon. Typically, in such a process, alumina powder is fed from a hopper with a fine mesh screen bottom into an oxygen stream. The oxygen flows downwardly through the central tube of an oxhydrogen burner. Hydrogen is fed to the flame through an annular passage surrounding the central tube. The flame from the burner is discharged into a furnace containing a ceramic pedestal. The pedestal is centered in the furnace below the bottom tip of the central tube of the burner, and at a predetermined vertical distance below said tip. The powder is fed intermittently through the central tube by tapping the hopper with a hammer. The powder falling through the flame builds up as a mass, either on the pedestal or on the tip of a thin corundum crystal supported on the pedestal. As the powder builds up on the upper surface of this mass, the pedestal is lowered in order to maintain a constant distance between the bottom tip of the central tube and the top of the growing crystal. The powder feed rate is adjusted at first so that the diameter of the growing crystal increases. When the diameter has attained the desired dimension, the powder feed rate is adjusted to maintain this diameter. When the flame temperature, powder feed rate, and pedestal withdrawal rate are properly coordinated, a cylindrical monocrystalline mass of corundum (e.g., alpha-alumina), known as a boule, is grown. The boule is then heat-treated or annealed at about 1900-1950° C. in a controlled atmosphere (e.g., oxidizing or reducing) for one to two days to help relieve stresses in the boule. Variations on this method are within the scope of the invention. In some embodiments, other powders are fed into the flame to produce other synthetic gemstones. In some embodiments, dopants are fed into the flame along with the gemstone material to produce colors or other effects in the resulting gemstone. Other suitable gemstone production methods, techniques, and/or processes understood by those in the field find use in embodiments of the present invention.

In some embodiments, boules (e.g., of synthetic ruby gemstone, of another gemstone, etc.) are cut, ground, polished, shaped, fashioned etc. into any suitable and/or desired shape and size. In some embodiments, synthetic gemstone material is shaped, cut, ground, polished, etc. with tooling (e.g., grinding wheel, drill, bit, blade, etc.) sufficient to remove material from the portions of synthetic gemstone object. In some embodiments, shaping, grinding, and/or cutting the synthetic crystals is carried out with diamond tooling. In some embodiments, graded diamond grit is affixed to a cutting surface by suitable methods. In some embodiments, diamond grit is overplated by nickel metal; as the tooling is used the surface of nickel is worn away leaving the sharp edge of diamond grit exposed, but held in place by the surrounding nickel metal. In some embodiments, diamonds are embedded in other metal and ceramic matrices. In some embodiments, grit (e.g., diamond grit) of varying grade (e.g., 24 . . . 30 . . . 40 . . . 50 . . . 75 . . . 100 . . . 150 . . . 300 . . . 600 . . . 900 . . . 1000), are used to shape, define and/or polish objects made from synthetic gemstone. In some embodiments, coarse grits (e.g., <100 grade) are used for grinding and shaping objects. In some embodiments, progressively finer grits (e.g., 200-800) are used for more precise shaping. In some embodiments, an object is finished with polishing size grits. In some embodiments, final polish is done with grit embedded in a soft matrix wheel (e.g., rubbers, felts, etc), or done in a batch polish method (e.g., resembling a rock tumbler with fine grit being pressed against the stones during agitation for an extended period).

In some embodiments, various cuts, grinds, etc. are made to produce objects of synthetic gemstone material in any desired shape, and with any desired size specifications. In some embodiments, precise (e.g., within 1 mm, within 0.1 mm, within 0.01 mm, within 1 μm) dimensions (e.g., radii, widths, lengths, heights, etc.) can be achieved using cutting and grinding methods described herein and/or understood in the field. In some embodiments, specialized tools and/or implements are used to create specific shapes out of the synthetic gemstone material (e.g., curves, flared ends, sharp corners, flat sides, etc.).

In some embodiments, the material is provided and finished so as to be non-porous and smooth so as to avoid any geometry that facilitates the accumulation of biological material or moisture that provide a growth environment for microorganisms.

In some embodiments, any synthetically-produced gemstone that has attractive properties for direct skin or tissue contact may find use in embodiments of the present invention. In some embodiments, synthetic gemstones that find use in embodiments of the present invention include, but are not limited to: alexandrite and other varieties of chrysoberyl, aquamarine and other varieties of beryl, chrysocolla, chrysoprase, diamond, emerald, feldspar (moonstone), garnet, hematit, jadeite and nephrite, jasper, kunzite, lapis lazuli, malachite, obsidian, olivine (peridot), opal (girasol), pyrite, quartz and its varieties (e.g., agate, amethyst (cardinal gem), citrine, chalcedony (bloodstone), onyx, tiger's-eye, etc.), ruby, sapphire, spinel, sugilite, tanzanite and other varieties of zoisite, topaz, turquoise, tourmaline, variscite, zircon, etc.

In some embodiments, any device, object, or composition that is placed in direct and/or continuous contact with the skin or tissue of a human or other animal finds use in embodiments, of the present invention. Exemplary devices, objects include, but are not limited to jewelry, watches, medical implants, dental implants, communication devices (e.g., blue tooth earpiece), entertainment devices (e.g., headphones, video game controllers), medical equipment, etc.

In some embodiments, the present invention provides objects consisting entirely of synthetic gemstone material. In some embodiments, the entire outer portion (e.g., exposed portion) of an object consists of synthetic gemstone material. In some embodiments, the entire portion of an object that contacts, is likely to contact, directly contacts, and/or continuously contacts human skin or tissue consists of synthetic gemstone material. In some embodiments, only portions of an object that must be made of another material to serve their function (e.g., carrying electric current, receiving a wireless communication, etc.) are not made of synthetic gemstone material. In some embodiments, the portions of objects that directly contact and/or continuously contact the skin consist entirely of synthetic gemstone material (e.g., earring back, inner portion of a ring, interior or back of a bracelet or necklace, beverage mouthpiece, contact points on an earpiece or headset, keys on a keyboard, handles (e.g., on a video game controller)).

In some embodiments, objects, devices, and compositions that contact tissue other than the top-most layers of skin present an enhanced risk of allergic reaction, infection, or other irritation or adverse effects. Examples of such objects, devices, and compositions include, but are not limited to: body piercing jewelry, medical implants (e.g., devices (e.g., pacemaker, etc.), pins, screws, plates, joints, valves, bone, etc.), dental implants (e.g., fake tooth, filling, retainer, braces).

In some embodiments, compositions of the present invention are configured for direct and continuous contact with the skin or tissue of a human or animal. In some embodiments, the duration of direct and continuous contact is: at least 10 minutes, at least 20 minutes, at least 1 hour, at least 2 hours, at least 4 hours, at least 8 hours, at least 12 hours, at least 1 day, at least 2 days, at least 10 days, at least 30 days, at least 1 year, indefinitely, or any duration sufficient to cause reaction, infection, and/or irritation. In some embodiments, the region of direct and continuous contact is at least 1 mm², at least 10 mm², 100 mm², at least 0.1 cm², at least 0.25 cm², at least 0.5 cm², at least 1 cm², at least 2 cm², or any region sufficient to cause reaction, infection, and/or irritation.

In some embodiments, skin and/or tissue contacted in embodiments of the invention include, but are not limited to: skin, epidermis, the cornified layer (stratum corneum), clear/translucent layer (stratum lucidum), granular layer (stratum granulosum), spinous layer (stratum spinosum), basal/germinal layer (stratum basale/germinativum), malpighian layer (stratum malpighi), cartilage, gingival, mucosal tissue, muscle tissue, bone, etc. In some embodiments, synthetic gemstone objects are configured for direct and continuous contact with the ear, earlobe, nose, nasal septum, face, eye brow, lip, tongue, nipple, genitals, foreskin, navel, mouth, gums, underlying muscle or tissue, or any other tissue or body part in which direct and continuous contact with an object is desired.

In some embodiments, the present invention provides jewelry consisting entirely of synthetic gemstone material. In some embodiments, such jewelry reduces the risk of irritation, allergic reaction, or infection. In some embodiments, jewelry is provided in which the portion of the jewelry that contacts human skin or tissue consists entirely of synthetic gemstone material. In some embodiments, such jewelry reduces the risk of irritation, allergic reaction, or infection. In some embodiments, the present invention is not limited by the type of jewelry or portion of jewelry that consists of synthetic gemstone material. In some embodiments, a clamp, pin, backing, plate, stud, ring, base, clasp, link, or any other portion of a piece of any type of jewelry consists of synthetic gemstone material.

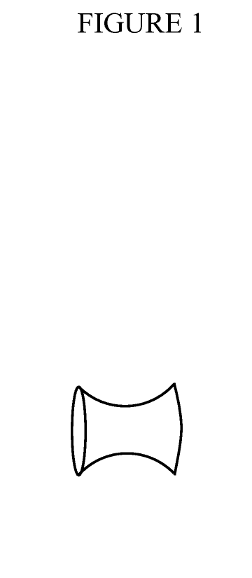

In some embodiments, jewelry entirely consisting of synthetic gemstone material includes, but is in not limited to body piercing or stretching: plugs, plates, bars, needles, pins, etc.; bracelets; necklaces; anklets, etc. In some embodiments, jewelry or other objects (e.g., stretchers) that are inserted in tunnels or fistula present risks for irritation. In some embodiments, the present invention provides objects (e.g., plugs, discs, plates, pins, bars, etc.) consisting entirely of synthetic gemstone material for insertion into natural or healed fistula (e.g., as jewelry). In some embodiments, such objects are designed, shaped, and or configured to fit (e.g., precisely) within such fistula. In some embodiments, a plug is cylindrical in shape. In some embodiments, a plug is a cylinder that is narrowed in the middle and flares out at either end (e.g., to maintain its position when inserted into a fistula or other tunnel) (SEE FIG. 1). In some embodiments, a plug, bar, pin, plate, etc. for skin or tissue stretching is 0.25 cm to 5 cm in length and 1 mm to 100 mm in diameter (e.g., 1 mm . . . 2 mm . . . 5 mm . . . 10 mm . . . 20 mm . . . 50 mm . . . 100 mm). In some embodiments, a plug, bar, pin, plate, etc. is between 1 and 100 mm in diameter at each end and 0.1 to 5 mm narrower in the middle. In some embodiments, the center diameter of a plug is 5-15% smaller than the end diameters. In some embodiments, stretchers are of any dimensions commonly used in the field, or necessary to provide the desired function within a particular piece of a human or animal anatomy.

In some embodiments, the present invention provides synthetic gemstone objects configured to fit on and/or within portions of a subject's body (e.g., naturally-occurring or manmade cavities). In some embodiments, synthetic gemstone objects are shaped and sized to contact and/or fit within specific body parts. For example, in some embodiments, the present invention provides ear plugs configured to fit through a fistula in the ear lobe, comprising, consisting essentially of, or consisting of synthetic gemstone material (e.g., synthetic ruby). In some embodiments, ear plugs comprise a cylinder-like shape with concave curves extending between the two disc-like ends. In some embodiments, an ear plug comprises cylinder-like shape with flared ends. In some embodiments, the specific radii of the grinding wheel used to shape the ear plug determines the shape and/or depth of the curve extending the length of the ear plug. In some embodiments, a curve and/or taper is large enough (e.g., deep enough) to hold the ear plug in the ear, but small enough to allow the tissue to stretch for insertion. In some embodiments, the center of the plug (that resides in the center of the fistula traversed by the plug) is less than 3 mm smaller than the flared end (e.g., 2 mm smaller . . . 1.8 mm smaller . . . 1.4 mm smaller . . . 1 mm smaller). In some embodiments, the larger the radius of the plug, the larger the size for the flare (e.g., difference between end radius and center radius). In some embodiments, similar plugs (e.g., of any suitable size) are configured to fit within cavities or fistula in other body parts. In some embodiments, similar shaping and sizing considerations are made for other objects configured for interaction with other body portions.

In some embodiments, the present invention provides methods for reducing the likelihood of allergic reaction, infection, or other irritation or adverse effects by replacing an existing device, object, or composition that is in direct and/or continuous contact with the skin or tissue with a similar device, object, or composition configured to place only synthetic gemstone in contact with skin or tissue.

We claim:

1. A body piercing plug consisting essentially of synthetic gemstone material, wherein said body piercing plug is configured for insertion into a fistula through human skin and tissue, and wherein said body piercing plug is configured to place said synthetic gemstone material in direct and continuous contact with human skin or tissue.

2. The body piercing plug of claim 1, wherein the region of direct and continuous contact comprises at least 0.5 cm² of surface area.

3. The body piercing plug of claim 2, wherein the region of direct and continuous contact comprises at least 1 cm² of surface area.

4. The body piercing plug of claim 1, wherein the duration of direct and continuous contact comprises at least 1 hour.

5. The body piercing plug of claim 1, wherein the duration of direct and continuous contact comprises at least 1 day.

6. The body piercing plug of claim 5, wherein the duration of direct and continuous contact comprises at least 1 week.

7. The body piercing plug of claim 6, wherein the body piercing plug is configured for insertion into a fistula through a human ear lobe.

8. The body piercing plug of claim 6, wherein the body piercing plug consists of synthetic gemstone material.

9. The body piercing plug of claim 1, wherein the body piercing plug comprises a cylinder-like shape that is narrowed in the middle and flares out at either end.

10. A method comprising inserting a body piercing plug of claim 1 into a fistula through human skin and tissue.

11. The method of claim 10, further comprising allowing the body piercing plug to remain in direct and continuous contact with the human skin or tissue.

12. The method of claim 11, wherein the fistula through human skin and tissue is a fistula through a human ear lobe

13. The method of claim 10, wherein inserting a body piercing plug of claim 1 provides reduced incidence of adverse reactions to the body piercing plug compared to body piercing plug comprising non-synthetic gemstone material.

14. The method of claim 10, wherein the region of direct and continuous contact comprises at least 0.5 cm² of surface area.

15. The method of claim 14, wherein the region of direct and continuous contact comprises at least 1 cm² of surface area.

16. The method of claim 10, wherein the duration of direct and continuous contact comprises at least 1 hour.

17. The method of claim 16, wherein the duration of direct and continuous contact comprises at least 1 day.

18. The method of claim 17, wherein the duration of direct and continuous contact comprises at least 1 week.

19. The method of claim 10, wherein the body piercing plug consists of synthetic gemstone material.

20. The method of claim 10, wherein the body piercing plug comprises a cylinder-like shape that is narrowed in the middle and flares out at either end.
